(19) United States
(12) Patent Application Publication
(10) Pub. No.: US 2026/0324958 A1
(43) Pub. Date: Oct. 22, 2026

(54) SPATIAL TRACKING OF TOOLS AND INSTRUMENTS IN AN OPERATING FIELD

(71) Applicant: Arthrex, Inc., Naples, FL (US)

(72) Inventors: Zachary Dominguez, Carpinteria, CA (US); John Batikian, Pismo Beach, CA (US); Andrew Melton, Santa Barbara, CA (US)

(73) Assignee: Arthrex, Inc., Naples, FL (US)

(21) Appl. No.: 19/099,555

(22) PCT Filed: Jul. 28, 2023
(86) PCT No.: PCT/IB2023/057711

§ 371 Date: Jan. 29, 2025

Related U.S. Application Data
(60) Provisional application No. 63/394,086, filed on Aug. 1, 2022.

(51) Int. Cl. A61B 1/005; A61B 1/00

(52) U.S. Cl. CPC A61B 1/009; A61B 1/00045; A61B 1/00097; A61B 1/00147

(57) ABSTRACT

A surgical apparatus includes a first surgical instrument, a second surgical instrument, and a flexible tether interconnecting the first surgical instrument to the second surgical instrument. The flexible tether includes a shape sensor configured to detect a path of the flexible tether and at least one rotational sensor configured to detect a rotation of the flexible tether. At least one controller is in communication with the flexible tether, wherein the controller is configured to identify a position and an orientation of the second surgical instrument relative to the first surgical instrument in response to the path and the rotation of the flexible tether.

### BACKGROUND

The present disclosure generally relates to a surgical imaging system and, more particularly, to an imaging apparatus that tracks a position of a surgical instrument for virtual display in a field of view.

Surgical imaging systems may be implemented to support various noninvasive operations. In such operations, endoscopes (e.g., arthroscopes, laparoscopes, etc.) may be utilized to provide visual feedback to surgeons and medical staff to guide and assist in locating internal features to treat patients. During such operations, endoscopes may be maneuvered in combination with various surgical instruments or devices to view and interact with the anatomy of patients. The disclosure provides for various systems and methods that may assist in the operation of such surgical imaging systems and instruments.

### SUMMARY

In various implementations, the disclosure provides for a surgical imaging system, apparatus, and related methods that implement one or more orientation or position sensors to track the position of surgical instruments in an operating field. A number of position tracking or orientation sensors may be implemented to track the position of the instruments relative to the camera or imaging apparatus. In particular, the disclosure may provide for surgical instruments to be tracked relative to a field of view of a camera. In this way, a controller, which may include a graphic processor and/or image processor, may monitor or track the position of the surgical instrument as it is manually oriented or maneuvered relative to the camera and the field of view. Based on the position and/or orientation of the surgical instrument within the field of view, the controller may generate and/or access a graphic rendering of the surgical instrument in the field of view and generate augmented image data for an image feed provided to a display to depict the graphic rendering of the surgical instrument. Such operation may be particularly beneficial in cases where the surgical instrument is occluded or blocked from the field of view of the camera.

In some implementations, a surgical apparatus may comprise a first surgical instrument, a second surgical instrument, and a flexible tether interconnecting the first surgical instrument to the second surgical instrument. The flexible tether may comprise a shape sensor configured to detect a path of the flexible tether and at least one rotational sensor configured to detect a rotation of the flexible tether. At least one controller is in communication with the flexible tether, wherein the controller is configured to identify a position and an orientation of the second surgical instrument relative to the first surgical instrument in response to the path and the rotation of the flexible tether.

In some implementations, the disclosure provides a method for controlling a surgical imaging system that includes capturing image data with a camera in a field of view and manually orienting a surgical device in connection with the camera via a flexible tether. A path of the flexible tether is decoupled from a rotation of the surgical device relative to the camera. The path of the flexible tether is identified via path data, and the rotation of the surgical device is identified relative to the camera via rotation data. A position and orientation of the surgical device relative to the camera is determined in response to the path data and the rotation data. An indication of a position and an orientation of the surgical device relative to the camera is output.

In some implementations, a surgical imaging system includes a camera configured to capture image data in a field of view, a manually oriented surgical instrument, and a flexible tether interconnecting the camera to the surgical instrument. The flexible tether comprises a plurality of orientation sensors configured to detect an orientation of the surgical instrument relative to the camera. At least one controller is in communication with the camera and the flexible tether. The controller is configured to identify a position and an orientation of the surgical instrument in response to orientation signals communicated from the orientation sensors and generate a graphic rendering of the surgical instrument projected in the field of view in response to the position and the orientation of the surgical instrument. An augmented image feed is output to a display demonstrating the image data with the graphic rendering.

These and other features, objects and advantages of the present disclosure will become apparent upon reading the following description thereof together with reference to the accompanying drawings.

### BRIEF DESCRIPTION OF THE DRAWINGS

FIG. 1 is an environmental view of a surgical imaging system demonstrating a tracking apparatus for a manually oriented surgical instrument;

FIG. 2 is a simplified schematic diagram of a surgical imaging system comprising a tracking apparatus;

FIG. 3 is a simulated representation of a position tracking environment modeling a position and orientation of a manually oriented surgical instrument depicted in image data of a surgical imaging system;

FIG. 4 is a flow chart demonstrating a method for tracking a position and orientation of a manually oriented surgical instrument and displaying augmented image data demonstrating a graphical representation of the surgical instrument in a field of view of a surgical imaging system; and

FIG. 5 is a modified block diagram of a surgical imaging system comprising a tracking apparatus in accordance with the disclosure.

### DETAILED DESCRIPTION OF THE PREFERRED EMBODIMENT

In the following description of the preferred implementations, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

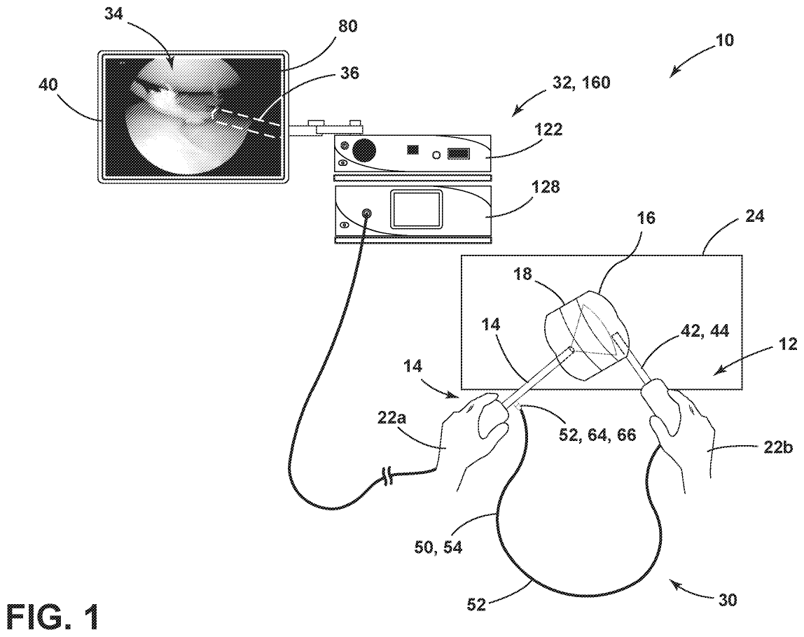

Referring to FIG. 1, the disclosure generally relates to apparatuses and corresponding methods for implementing a surgical imaging system 10 configured to track a position and/or orientation of one or more manually oriented surgical instruments 12. In an exemplary embodiment, the surgical imaging system 10 may comprise an endoscope 14 or camera configured to capture image data within a field of view 16. As shown in FIG. 1, the field of view 16 of the endoscope 14 may capture image data representing an orifice or cavity 18 within the anatomy of a patient. In such applications, maneuvering the endoscope 14 to position the field of view 16 to view the manually oriented surgical instrument 12 may require extensive experience as well as advanced relative spatial positioning between a left hand 22a and right hand 22b of a user (e.g., a surgeon, physician, medical professional). Additionally, in some instances or procedures, portions of the surgical instruments 12 may be partially or completely occluded in the field of view 16 of an operating field 24, such that the corresponding image data does not depict the position, orientation, or operating characteristics of the manually oriented surgical instrument 12. In some cases, blind or occluded operation of surgical instruments 12 may be inherent to a surgical procedure as being necessary to access portions of the anatomy of the patient that are not visible within the field of view. Though such practices can be perfected and can provide highly beneficial, noninvasive techniques to promote improved patient outcomes, the practice of such techniques may require considerable experience and practice.

In various implementations, the disclosure provides for a tracking device or a tracking apparatus 30 that detects and monitors the position and orientation of the one or more manually oriented surgical instruments 12 relative to the field of view 16 of an endoscope 14 or camera. By tracking the position and orientation of the surgical instrument 12, a controller 32 of the system 10 may be operable to generate augmented image data 34, including a graphic representation 36 of the surgical instrument 12 in the field of view 16 as presented on a display device 40. In this way, the surgical imaging system 10 may present the augmented image data 34 to the user, such that the position of the surgical instrument 12 relative to the field of view 16 may be readily identified on the display device 40 even when the surgical instrument 12 is occluded from view in the image data.

As introduced in reference to FIG. 1 and further discussed in the following detailed description, the disclosure may provide for the surgical imaging system 10 to provide advantageous tracking functions in relation to the position and the orientation of handheld or manually oriented instruments, exemplified as the surgical instrument 12. As described herein, surgical instruments 12 may correspond to various forms of powered or manually actuated surgical or medical devices, instruments, or implements that may be utilized in conjunction with the surgical imaging system 10. Accordingly, any instruments, devices, and/or implements that may be cataloged or modeled in a memory or database in communication with the controller 32 may be modeled as graphic representations 36 in the augmented image data and tracked by the tracking apparatus 30. Based on the graphic representations 36 and position data from the tracking apparatus 30, the controller 32 may accurately position, spatially or perspectively modify, and demonstrate the representations 36 of the instruments, devices, and/or implements on the display device 40. Examples of surgical instruments 12 or devices that may be implemented with the surgical imaging system 10 include, but are not limited to, a biter, a grasper, a retriever, a pick, a punch, a scalpel, a hook, a probe, scissors, a retractor, or various surgical instruments. Additional exemplary instruments 12 or devices may include one or more powered surgical accessories or tools including, but not limited to, ablation probes, drills, saws, burrs, cutting blades, rasps, shavers, and various additional resection or power surgical tools.

In various implementations, the surgical instrument 12 may comprise one or more interchangeable accessories 42, each of which may include different operating functions, as well as different shapes and dimensions. Accordingly, to support the presentation of the augmented image data 34 and the corresponding graphic representation 36 of each of the surgical devices or instruments 12, the controller 32 of surgical imaging system 10 may access corresponding models (e.g., three-dimensional models) of the surgical instruments 12. Based on the corresponding models of each of the surgical instruments, the controller 32 may identify the position and orientation of the surgical instrument 12 based on tracking data communicated from the tracking apparatus 30. The controller 32 may then adjust the position and orientation of the graphic representation 36 of the surgical instrument 12 by manipulating the corresponding model. In this way, the controller 32 may generate the augmented image data 34 for presentation on the display device 40.

In various implementations, the level of detail or corresponding information communicated or presented in the graphic representation 36 of the surgical instrument 12 may vary based on the particular application. For example, as demonstrated in FIG. 1, the augmented image data 34 includes a simple outline demonstrating the position and orientation of the surgical instrument 12 to communicate the graphic representation 36. In some examples, additional details and dimensionally accurate features of the surgical instrument 12 may be represented in the graphic representation 36. Accordingly, the level of detail and sophistication of the graphic representation 36 demonstrated in the augmented image data 34 may be scaled or adjusted to suit various applications of the surgical imaging system 10.

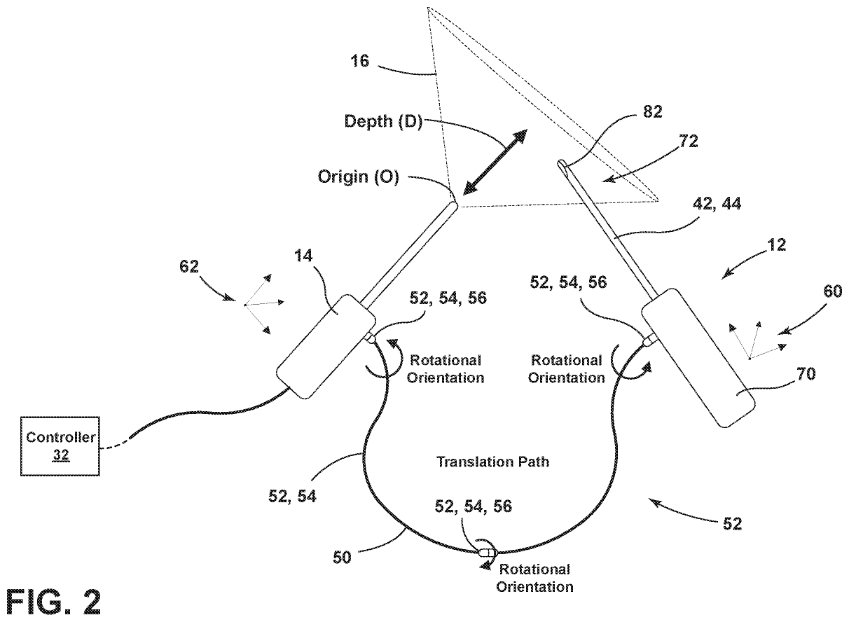

Referring now to FIGS. 1, 2, and 3, exemplary details of the tracking apparatus 30 are demonstrated and discussed in reference to the generation and presentation of the augmented image data 34. As demonstrated in FIG. 2, the tracking apparatus 30 is demonstrated as a flexible tether 50 interconnecting the camera or endoscope 14 to the surgical instrument 12. In an exemplary embodiment, the flexible tether 50 may incorporate one or more orientation or position sensors, which may generally be referred to as tracking sensors 52. As shown, the tracking sensors 52 may extend along a length of the flexible tether 50 between the endoscope 14 and the surgical instrument 12 and/or interconnect one or more segments or portions of the flexible tether 50. As described in various examples, the tracking sensors 52 may be implemented in a variety of combinations and may include various sensory technologies.

In some implementations, the tracking sensors 52 may comprise one or more shape sensors 54 extending along and incorporated in the length of the flexible tether 50. For example, the shape sensors 54 or an array of shape sensors 54 may correspond to one or more strain sensors distributed along the length of the flexible tether 50. In operation, the controller 32 may receive signals from the shape sensors 54 and identify the three-dimensional shape or translational path of the flexible tether 50 extending between the endoscope 14 and the instrument 12 as the summation of each of the bending or strain components identified by the shape sensors 54 along the length of the flexible tether 50. In some examples, the shape sensor or sensors 54 may correspond to a multi-core optical fiber comprising a plurality of fiber Bragg grading sensors. In operation, the fiber Bragg grading sensors or optical sensors extending along the length of the tether 50 may operate to detect a bending extent or curvature of the flexible tether 50 as well as a direction of the curvature based on signals received from the shape sensors 54 distributed along the length of the flexible tether 50. In this way, the tracking apparatus 30 of the imaging system 10 may identify a spatial relationship (e.g., position and orientation) between a first coordinate system or instrument coordinate system 60 of the surgical instrument 12 and a second coordinate system or imager coordinate system 62. By monitoring the spatial relationship between the instrument coordinate system 60 and the imager coordinate system 62, the controller 32 may detect changes in the relative position and orientation of the surgical instrument 12 relative to the field of view 16 of the endoscope 14. Based on the spatial relationship between the surgical instrument 12 and the endoscope 14, the controller 32 may generate the graphic representation 36 and present the augmented image data 34 on the display device 40 to assist in the operation of the surgical imaging system 10.

In some implementations, the tracking apparatus 30 may incorporate one or more of the tracking sensors 52 in the form of rotation sensors 64. In such configurations, the rotation sensors 64 may comprise one or more rotational linkages 66 (e.g., swiveling linkages, rotational bearing linkages, etc.) interconnecting portions of the flexible tether 50 to the endoscope 14, the surgical instrument 12, or segments along the length of the flexible tether 50. In operation, the rotational linkage 66 may rotationally decouple the rotation of the surgical instrument 12 relative to the endoscope 14, such that the one or more rotational linkages 66 provide for free rotation between the surgical instrument 12 and the endoscope 14 without resulting in torsion along the length of the flexible tether 50. In response to the rotation of the rotational linkage 66, the one or more rotation sensors 64 may detect and report changes in rotational orientation of the rotational linkage 66 to the controller 32. Accordingly, when applied in combination with the shape sensors 54 or fiber optic shape sensors, the tracking sensors 52 may enable the controller 32 to track the relative position of the surgical instrument 12 relative to the endoscope 14 based on variations in a translational path extending along the length of the flexible tether 50 as well as the orientation of the surgical instrument 12. In this way, the surgical instrument 12 may be manually oriented freely relative to the field of view 16 of the endoscope 14 while the tracking apparatus 30 detects and monitors the spatial relationship between the instrument coordinate system 60 and the imager coordinate system 62.

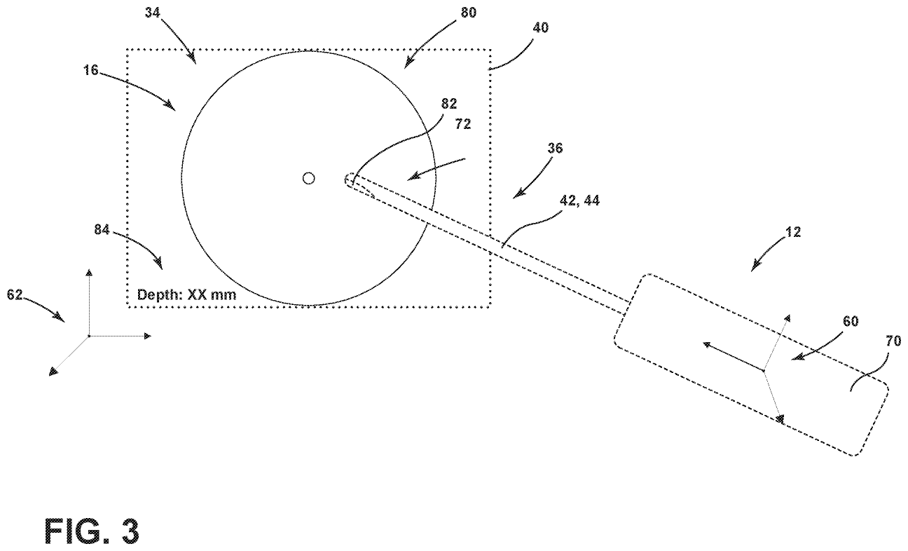

Still referring to FIG. 2, the surgical instrument 12 is demonstrated comprising an interchangeable accessory 42 extending from a handle portion 70 to a distal end portion 72. As generally discussed previously, the dimensional proportions of each of the surgical instruments 12 in combination with the interchangeable accessory 42 may be accessed by the controller 32 in the form of model information for each combination. The specific style, type, or identification associated with the instruments 12 and accessories 42 may be identified in response to a connection or communication between the surgical instrument 12 and the controller 32, which may be transmitted wirelessly or through a wired connection (e.g., via the flexible tether 50). Once the surgical instrument 12 and connected accessory 44 are identified, the controller 32 may access model information (e.g., dimensions, proportions, features, etc.) of the surgical instrument 12 and the connected accessory 44 from a memory or connected server or database in communication with the controller 32. Based on the model information of the surgical instrument 12 and the connected accessory 44, the controller 32 may generate the graphic representation 36 of the surgical instrument 12 and the connected accessory 44 to generate the augmented image data 34 as exemplified in FIG. 3.

Referring now to FIGS. 2 and 3, the graphical representation 36 of the surgical instrument 12, as well as the connected accessory 44, may be demonstrated over the extent of the field of view 16 as well as a display portion corresponding to a perimeter mask or field-stop mask 80 presented on the display device 40. As shown, the graphic representation 36 may include representations of one or more features 82 or operating features (e.g., actuators, blades, acting ends, points, protrusions, cutting edges, suction inlets, supply outlets, etc.) that may be specific to the surgical instrument 12 and/or the connected accessory 44. In various implementations, the graphic representation 36 may be generated and updated by the controller 32 to demonstrate the relative orientation of each of the features 82 as well as their corresponding position in the field of view 16 and/or the surrounding area demonstrated by the field-stop mask 80. In this way, the graphic representation 36 of the surgical instrument 12 may present the relative orientation of each of the features 82 of the surgical instrument 12 and/or the connected accessory 44 within the field of view 16 to inform the user of the corresponding position and orientation.

In some implementations, the controller 32 may further present a depth indication 84 of the surgical instrument 12 or one or more features 82 of the surgical instrument 12 on the display device 40. For example, during a blind-access step of a surgical procedure, a physician may identify one or more of the features 82 of the surgical instrument 12 and/or the connected accessory 44 to be monitored and presented on the display device 40. The controller 32 may identify and communicate the depth indication 84 of each of the operating features 82 based on the proportions or dimensions of the surgical instrument 12 and/or connected accessory 44 based on the programmed locations or dimensions in the instrument coordinate system 60. Accordingly, the controller 32 may track the relative position of each of the operating features 82 of the surgical instrument 12 in the imager coordinate system 62 by monitoring the spatial relationship between the endoscope 14 and the surgical instrument 12 with the tracking apparatus 30. In this way, the graphic representation 36 may be scaled based on a relative depth D or proximity of the surgical instrument 12 to an origin O or zero depth of focal distance of the field of view 16 of the camera or imager of the endoscope 14. Accordingly, the controller 32 may include a numeric or symbolic indication in the form of the depth indication 84, scale, color-coded depth indication, and/or scale the graphic representation 36 of the surgical instrument 12 in the augmented image data 34. The depth indication may indicate the relative depth of the surgical instrument 12 and/or the connected accessory 44 and their corresponding operating features 82, even in cases when the surgical instrument 12 is occluded from the field of view 16. Accordingly, the proximity of the surgical instrument 12 may be communicated even in cases where the physical representation of the surgical instrument 12 is hidden by one or more additional surgical implements or instruments or portions of the anatomy of the patient.

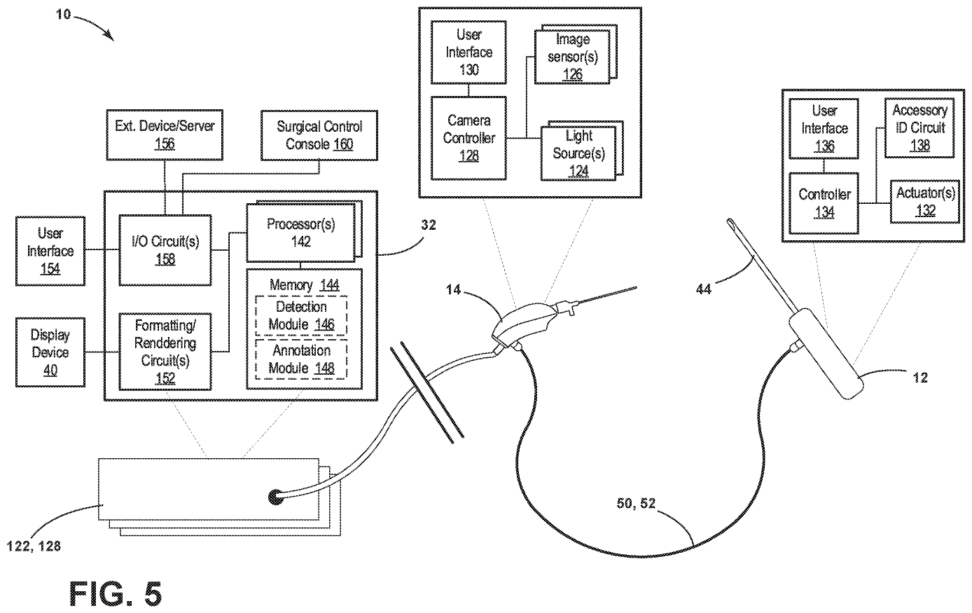

In some implementations, the controller 32 may automatically activate and deactivate the presentation of the graphic representation 36 in the augmented image data 34. For example, the image data captured by the camera or imager of the endoscope 14 may be processed by one or more imaging circuits or processors of the controller 32 to identify one or more features or objects within the image data. In an exemplary embodiment, the controller 32 may be configured to detect the presence of one or more portions of the surgical instrument 12 and/or the connected accessory 44 in the image data via various recognition or processing algorithms. Further discussion regarding the image processing and corresponding processors of the controller 32 implemented to operate the surgical imaging system 10 are discussed in reference to the block diagram demonstrated in FIG. 5.

In operation, the controller 32 may selectively activate or present the graphic representation 36 of the surgical instrument 12 in response to one or more conditions, which may comprise: a determination that one or more portions of the surgical instrument 12 are located within the field of view 16; and/or a determination that the surgical instrument 12 is not visible or is at least partially occluded within the image data captured by the endoscope 14 in the field of view 16. In cases where the surgical instrument is located within the field of view 16 as detected by the tracking apparatus 30, the controller 32 may continue to determine whether or not the surgical instrument 12 is visible within the field of view 16. If the surgical instrument 12 is not visible or is at least partially occluded within the image data captured in the field of view 16, the controller 32 may overlay one or more portions of the image data with the graphic representation 36 of the surgical instrument 12 to generate the augmented image data 34. In some cases, the graphic representation 36 may depict the features 82 of the surgical instrument 12 as represented in the image data, which may allow a user to better determine the relative orientation demonstrated in the field of view 16. In some cases, only portions of the graphic representation 36 may be displayed in the augmented image data 34, which may be superimposed or overlaid on portions of the image data where the surgical instrument is occluded from view. Accordingly, the surgical imaging system 10 may provide for a flexible solution that may be tailored to suit the needs and preferences of various users.

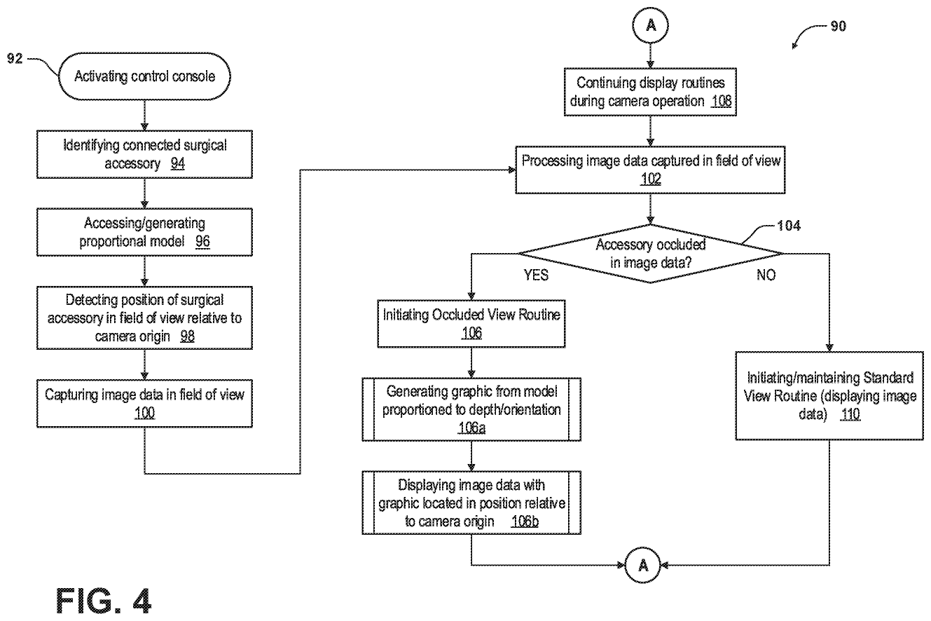

Referring now to FIG. 4, a flow chart is shown demonstrating a control routine or method 90 for the surgical imaging system 10. The method 90 may begin in response to the activation of the control console or the activation of the controller 32 of the surgical imaging system 10 (92). Once the console is activated, the controller 32 may interrogate one or more I/O ports to determine a connection status of the surgical instrument 12 and/or the connected accessory 44 (94). In response to a communication from the surgical instrument 12 or accessory 44, the controller 32 may identify the surgical instrument 12 and/or connected accessory 44 (e.g., a model number, serial number, device type, etc.)(96). Based on the identity of the surgical instrument 12, the controller 32 may access model information or dimensional information for the surgical instrument 12, such that the graphic representation 36 may be generated for display as the augmented image data 34 (98).

Upon activation of the control console as discussed in reference to step 92 in combination with the detection of the surgical instrument 12, the controller 32 may further detect and monitor a relative position of the surgical instrument 12 relative to the endoscope 14 (98). As previously discussed, the controller 32 may track the specific proportions of the surgical instrument 12 and/or the connected accessory 44 by monitoring the orientation and position of the instrument coordinate system 60 relative to the imager coordinate system 62 with the tracking apparatus 30. In addition to the detection of the position of the surgical instrument 12 and the accessing of the model data for the graphic representation 36, the controller 32 may further activate a camera or imager of the endoscope 14 to capture image data in the field of view 16 (100). Based on the image data, the controller 32 may process the features identified in the field of view 16 to determine if the surgical instrument 12 and/or the connected accessory 44, as well as the corresponding operating features 82 or other features of interest, are identified (102). Based on the image processing, the controller 32 may continue to determine if one or more portions of the surgical instrument 12 are occluded from view in the image data presented in the field of view 16 (104).

If in step 104 the controller 32 determines that the surgical instrument 12 is occluded or partially occluded within the image data, the controller 32 may initiate an occluded view control routine (106). The occluded view control routine 106 may include steps of generating the graphic representation 36 from the model data of the surgical instrument 12 and/or the connected accessory 44 and proportioning and positioning the graphic representation 36 in the field of view 16 (106a). Based on the graphic representation 36, the controller 32 may continue to display the augmented image data 34, including the graphic representation 36 located in the position and orientation relative to the image or coordinate system 62 and the field of view 16 (106b). Throughout operation of the image processing control routine or method 90, the controller 32 may continue to monitor and process the image data to determine if the surgical instrument 12 is partially or completely occluded, as demonstrated by step 108, which returns the method 90 to step 102.

If in response to processing of the image data the controller 32 identifies that the surgical instrument 12 is not occluded in the image data, the controller 32 may initiate or maintain a standard viewing routine that may display the image data as captured in the field of view 16 without the graphic representation 36 (110). In some implementations, the method 90 or various embodiments of the imaging surgical system 10 may also or alternatively provide for the manual activation or display of the augmented image data 34. For example, the user may selectively activate the display of the graphic representation 36 and generation of the augmented image data 34 in response to one or more inputs into a user interface of the surgical instrument 12, the endoscope 14, and/or one or more control consoles or controllers associated with the surgical imaging system 10. Accordingly, the surgical imaging system 10 may be flexibly implemented to suit a variety of applications.

Referring now to FIG. 5, a block diagram of the imaging system 10 is shown. As discussed throughout the disclosure, the system 10 may comprise the camera or endoscope 14 in communication with a display controller 122, which may be implemented as a component of the controller 32 or as a discrete device. The endoscope 14 may comprise a plurality of light sources 124, an image sensor 126, a camera controller 128, and a user interface 130. In various implementations, the endoscope 14 may correspond to an arthroscope with an elongated rigid body configured to access various cavities or orifices of the patient for noninvasive surgical procedures. For example, the distal end may include a diameter of less than 2 mm. As demonstrated, the endoscope 14 may be in communication with the display controller 122 via a communication interface. Though shown connected via a conductive connection, the communication interface may correspond to a wireless communication interface operating via one or more wireless communication protocols (e.g., Wi-Fi, 802.11 b/g/n, etc.).

The light source 124 may correspond to various light emitters configured to generate light in the visible range and/or the near infrared range. In various implementations, the light source(s) 124 may include light emitting diodes (LEDs), laser diodes, or other lighting technologies. The image sensor 126 may correspond to be implemented in a variety of configurations comprising, for example, charge-coupled devices (CCD) sensors, complementary metal-oxide semiconductor (CMOS) sensors, or similar sensor technologies. As previously discussed, the system 10 (e.g., the display controller 122) may process the image data to detect the presence of the surgical instrument 12 and/or the connected accessory 44. Based on the presence or occlusion of one or more portions of the surgical instrument 12, the controller 122 or, more generally, the controller 32 may selectively control the display of the graphic representation 36. Additionally, the display controller 122 may manipulate the model data of the surgical instrument 12 and/or the connected accessory 44 to generate the graphic representation 36 in the location and orientation identified by the tracking apparatus 30.

The camera controller 128 may correspond to a control circuit configured to control the operation of image sensor 126, the light sources 124, or various other features of the endoscope 14. In various implementations, the camera controller 128 may be in communication with a user interface 130, which may include one or more input devices, indicators, displays, etc. The user interface 130 may provide for the control of the endoscope 14, including the activation of one or more routines as discussed herein. As discussed herein, the controllers (e.g., controller 32, controller 128, etc.) may be implemented with one or more general purpose processors, microprocessors, microcontrollers, application specific integrated circuits (ASIC), field programmable gate arrays (FPGAs), a computational processing units, graphic processing units, a group of processing components, or other suitable electronic processing components.

The surgical implement or instrument 12 may correspond to a manually oriented device (e.g., handheld, hand or head-mounted, mounted to a handheld device, etc.) that is spatially manipulated by a portion of a user. In some implementations, the instrument 12 may correspond to a handheld tool that may be manually actuated or powered by connected device(s) or actuator(s) 132 (e.g., a motorized cutting tool, electro-surgical or ablation probe, etc.). Examples of surgical instruments 12 or devices that may be implemented with the surgical imaging system 10 include, but are not limited to, a biter, a grasper, a retriever, a pick, a punch, a scalpel, a hook, a probe, scissors, a retractor, or various surgical instruments. Additional exemplary instruments 12 or devices may include one or more powered surgical accessories or tools including, but not limited to, ablation probes, drills, saws, burrs, cutting blades, rasps, shavers, and various additional resection or power surgical tools. Accordingly, some instruments may include one or more control circuits 134 that may control the operation of such devices or instruments 12 in response to control signals from the controller 32 or an associated user interface 136 (e.g., buttons, switches, levers, detection sensors, etc.).

In some implementations, the control circuit(s) 134 of the instrument 12 may be in communication with the controller 32 and may further comprise an identification circuit 138. The identification circuit 138 may be configured to detect or identify the connected accessory 44 or the instrument 12 and communicate an identity or configuration (e.g., a device identification code, serial number, product code, etc.) of the instrument 12 and/or the connected accessory 44 to the controller 32. The controller 32 may utilize the identity of the instrument 12 and/or the connected accessory 44 to access model data for various surgical implements 12 and interchangeable accessories 42 in order to generate the graphic representation 36 and present the augmented image data 34 on the display device 40. The identification circuit 138 may communicate the identity or configuration of the instrument 12 and/or the connected accessory 44 to the controller 32 of the system 10 via the control circuit 134 and/or direction via a wired or wireless communication interface. For example, the identification circuit 138 or control circuit 134 may communicate the identity or device configuration of the instrument 12 and/or the connected accessory 44 via a variety of wired communication protocols (e.g., serial, Universal Serial Bus (USB), Universal Asynchronous Receiver/Transmitter (UART), etc.) and/or a wireless communication interface (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods.

The display controller 122 may comprise a processor 142 and a memory 144. The processor 142 may include one or more digital processing devices including, for example, a central processing unit (CPU) with one or more processing cores, a graphic processing unit (GPU), digital signal processors (DSPs), field programmable gate arrays (FPGAs), application specific integrated circuits (ASICs), and the like. In some configurations multiple processing devices are combined into a System on a Chip (SoC) configuration while in other configurations the processing devices may correspond to discrete components. In operation, the processor 142 executes program instructions stored in the memory 144 to perform the operations described herein.

The memory 144 may comprise one or more data storage devices including, for example, magnetic or solid state drives, and random access memory (RAM) devices that store digital data. The memory 144 may include one or more stored program instructions, object detection templates, image processing algorithms, etc. As shown, the memory 144 may comprise a detection module 146 and a rendering and augmentation module 148. The detection module 146 includes instructions to process the image data to detect and/or identify various features or portions of the surgical instrument 12. In some cases, the detection module 146 may include instructions to detect or identify a type or classification associated with the surgical instrument 12 in the image data captured by the endoscope 14. For example, the processor 142 may access instructions in the detection module 146 to perform various processing tasks on the image data including preprocessing, filtering, masking, cropping, and various enhancement techniques to improve detection capability and efficiency. Additionally, the detection module 146 may provide instructions to process various feature detection tasks, including template matching, character recognition, feature identification or matching, etc. In some examples, the detection module 146 may also include various trained models for object detection and/or labeling surgical implements 12 or related objects. In some implementations, the detection of a surgical instrument or implement, either by identity, presence, or classification, may be monitored to initiate the control of display of the graphic representation 36 in the augmented image data 34. Once generated, the display controller 122 may output the augmented image data 34 on the display device 40 for operation with the surgical imaging system 10.

The rendering and augmentation module 148 may comprise instructions indicating various marking or overlay options to generate the augmented image data 34 as well as corresponding display filters to superimpose or apply the graphic representation 36 as an overlay to the image data demonstrating the field of view 16. As previously discussed, the augmented image data 34 may also include one or more graphics, annotations, labels, markers, and/or identifiers that indicate the depth D, location, presence, identity, or other information related to a classification or identification of the surgical instrument 12. The rendering and augmentation module 148 may further provide instructions to generate graphics, labels, overlays, or other associated graphical information that may be applied to the image data captured by the camera or endoscope 14. In order to generate the graphic representation projected to accurately reflect the proportions and orientation of the surgical instrument 12 in the field of view 16, the display controller 122 may comprise a graphic processing engine or graphic processing unit (GPU) (not shown). The GPU may be in the form of one or more special-purpose processors configured to manipulate three-dimensional graphic data.

The display controller 122 may further comprise one of more formatting circuits 152, which may process the image data received from the endoscope 14, communicate with the processor 142, and output the augmented image data 34 to the display device 40. The formatting circuits 152 may include one or more signal processing circuits, analog-to-digital converters, digital-to-analog converters, etc. The display controller 122 may comprise a user interface 154, which may be in the form of an integrated interface (e.g., a touchscreen, input buttons, an electronic display, etc.) or may be implemented by one or more connected input devices (e.g., a tablet) or peripheral devices (e.g., keyboard, mouse, etc.). As shown, the controller 32 is also in communication with an external device, database, or server 156, which may correspond to a network, local or cloud-based server, device hub, central controller, or various devices that may be in communication with the display controller 32 and, more generally, the imaging system 10 via one or more wired (e.g., Ethernet) or wireless communication (e.g., Wi-Fi, 802.11 b/g/n, etc.) protocols. For example, the display controller 122 may receive updates to the various modules and routines as well as access model data for various surgical implements 12 and interchangeable accessories 42 from a remote server for improved compatibility, operation, diagnostics, and updates to the system 10. The user interface 154, the external server 156, and/or the surgical control console 160 may be in communication with the controller 32 via one or more I/O circuits 158. The I/O circuits 158 may support various communication protocols including, but not limited to, Ethernet/IP, TCP/IP, Universal Serial Bus, Profibus, Profinet, Modbus, serial communications, etc.

As previously discussed, the controller 32 may further be in communication with the tracking sensors 52 of the tracking apparatus 30. In some examples, the tracking apparatus 30 may be implemented as a discrete device in communication with the display controller 122 or may be integrated in a common console as a portion of the controller 32. Accordingly, the tracking apparatus 30 may communicate the position information identifying the spatial relationship or relative position of the surgical instrument 12 and instrument coordinate system 60 relative to the field of view 16 of the endoscope or imager coordinate system 62. In order to accurately and efficiently monitor the spatial relationship, the tracking apparatus may include various filters, conditioning circuits, processors, arithmetic logic units (ALUs), digital signal processing units (DSPs), and various other processing circuits.

The tracking sensors 52 may include a variety of sensor technologies that may track the shape of the flexible tether 50 as well as the orientation of the surgical instrument 12 or the endoscope 14 relative to each other or the operating environment. For example, the tracking sensors 52 may include one or more optical shape sensors (e.g., fiber Bragg grating shape sensors, sensor arrays, strain gauges, etc.). Additionally, the tracking sensors 52 may include one or more rotary positions or rotation sensors 64 configured to detect the relative rotational position of the surgical instrument 12 relative to the endoscope 14. In some implementations, the orientation of the surgical instrument 12 relative to the endoscope 14 and the corresponding field of view 16 may be tracked relative to the operating environment (e.g., relative to gravity, a reference signal or point, a compass direction, a beacon or spatial artifact, etc.). In various implementations, the orientation of the surgical instrument 12 relative to the endoscope 14 may be determined in by the tracking apparatus 30 based on a comparison of the orientation of the instrument coordinate system 60 to the imager coordinate system 62. Based on the spatial relationship between the instrument coordinate system 60 and the imager coordinate system 62, the controller 32 (e.g., the display controller 122) may accurately determine the position and orientation of the surgical instrument relative to the field of view 16 and present the graphic representation 36 of the surgical instrument 12 in the augmented image data 34 having an orientation and proportions representative of the surgical instrument 12 projected in the image data captured by the endoscope 14.

According to some aspects of the disclosure, a surgical apparatus comprises a first surgical instrument, a second surgical instrument, and a flexible tether interconnecting the first surgical instrument to the second surgical instrument. The flexible tether includes a shape sensor configured to detect a path of the flexible tether and at least one rotational sensor configured to detect a rotation of the flexible tether. At least one controller is in communication with the flexible tether. The controller is configured to identify a position and an orientation of the second surgical instrument relative to the first surgical instrument in response to the path and the rotation of the flexible tether.

According to various aspects, the disclosure may implement one or more of the following features or configurations in various combinations:
the shape sensor comprises an optical fiber sensor having a plurality of segments rotationally decoupled by the at least one rotation sensor;
at least one rotation sensor interconnects adjacent segments of the plurality of segments and measures the rotation as a rotational relationship between the adjacent segments;
at least one rotational sensor comprises a rotational linkage that rotationally decouples the flexible tether from at least one of the first surgical instrument and the second surgical instrument;
at least one controller is configured to track the rotation identified by the rotation sensor from the at least one rotational sensor independent of the path of the shape sensor;
at least one of the first surgical instrument and the second surgical instrument is a manually manipulated device;
the manually manipulated device is manually oriented to a portion of a user;
the second surgical instrument moves freely relative to the first surgical instrument within a boundary defined by an extent of the flexible tether in connection with the first surgical instrument;
the first surgical instrument comprises a camera configured to capture image data in a field of view;
at least one controller is further configured to identify a position and orientation of the surgical instrument in the field of view of the camera in response to the path and the rotation of the flexible tether;
at least one controller is further configured to generate a graphic rendering of the surgical instrument projected in the field of view in response to the position and the orientation of the surgical instrument and output an augmented image feed to a display demonstrating the image data with the graphic rendering;
the second surgical instrument comprises at least one of a biter, grasper, retriever, pick, punch, hook, probe, elevator, retractor, a cutting blade or scissors; and/or
the second surgical instrument is a surgical power tool comprising at least one of an ablation probe, a drill, a saw, a burr, a rasp, and a shaver.

According to another aspect of the disclosure, a method for controlling a surgical imaging system comprises capturing image data with a camera in a field of view and manually orienting a surgical device in connection with the camera via a flexible tether. The method then decouples a path of the flexible tether from a rotation of the surgical device relative to the camera. The path of the flexible tether is identified via path data, and the rotation of the surgical device relative to the camera id identified via rotation data. A position and an orientation of the surgical device relative to the camera are determined in response to the path data and the rotation data.

According to various aspects, the disclosure may further implement one or more of the following features or configurations in various combinations:
the path is identified based on a plurality of rotationally decoupled path segments of the flexible tether;
the rotation is identified based on a rotational relationship between or among the rotationally decoupled path segments;
the rotation is identified by measuring a rotational relationship between adjacent portions of the rotationally decoupled path segments;
the indication of the position and the orientation comprises a location of the surgical device relative to the field of view;
generating a graphic rendering of the surgical device projected in the field of view in response to the position and the orientation of the surgical device and outputting an augmented image feed to a display demonstrating the image data with the graphic rendering;
detecting a presence of the surgical device in the image data in response to the position and the orientation;
determining that the surgical device is occluded in the field of view based on the position and orientation of the surgical device and the presence of the surgical device in the image data; and/or
selectively generating an augmented image feed with at least one graphic portion of the graphic rendering overlaid on the at least one occluded portion.

According to yet further aspects of the disclosure, a surgical imaging system comprises a camera configured to capture image data in a field of view, a manually oriented surgical instrument, and a flexible tether interconnecting the camera to the surgical instrument. The flexible tether comprises a plurality of orientation sensors configured to detect an orientation of the surgical instrument relative to the camera. At least one controller is in communication with the camera and the flexible tether. The controller is configured to identify a position and an orientation of the surgical instrument in response to orientation signals communicated from the orientation sensors, generate a graphic rendering of the surgical instrument projected in the field of view in response to the position and the orientation of the surgical instrument, and output an augmented image feed to a display demonstrating the image data with the graphic rendering.

According to various aspects, the disclosure may implement one or more of the following features or configurations in various combinations:
the controller is configured to identify whether the surgical instrument is positioned in the field of view based on the position and the orientation;
the controller is configured to detect a presence of the surgical instrument in the image data;
the controller is further configured to determine if the surgical instrument is occluded in the field of view based on the position and orientation of the surgical instrument and the presence of the surgical instrument in the image data;
the controller is further configured to identify at least one occluded portion of the surgical instrument in the image data and selectively generate the augmented image feed with at least one graphic portion of the graphic rendering overlaid on the at least one occluded portion;
the surgical instrument comprises a connected accessory of a plurality of interchangeable accessories;
the controller is configured to identify an accessory identification of the connected accessory;
the controller is further configured to, in response to the accessory identification, access a graphic model of the connected accessory and generate the graphic rendering of the connected accessory based on the graphic model;
the plurality of interchangeable accessories comprise surgical devices having a variety of proportions, each represented by the graphic model accessed in response to the accessory identification;
the graphic rendering is generated demonstrating a projected view of the connected accessory in the field of view based on the position and the orientation;
at least one orientation sensor comprises an optical fiber extending along the flexible tether;
the controller is further configured to identify the position of the surgical instrument relative to the camera by identifying a translational path of the optical fiber from the camera to the surgical instrument;
at least one orientation sensor corresponds to a plurality of orientation sensors further comprising at least one rotational sensor in connection with the flexible tether;
at least one rotational sensor detects a rotational orientation of the surgical instrument relative to the camera; and/or
at least one rotational sensor comprises a rotational linkage that rotationally decouples the flexible tether from the camera and the surgical instrument, such that the rotation of the surgical instrument is tracked independent of a translation path of the optical fiber.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical apparatus comprising:
a first surgical instrument;
a second surgical instrument;
a flexible tether interconnecting the first surgical instrument to the second surgical instrument, wherein the flexible tether comprises a shape sensor configured to detect a path of the flexible tether and at least one rotational sensor configured to detect a rotation of the flexible tether; and
at least one controller in communication with the flexible tether, wherein the controller is configured to identify a position and an orientation of the second surgical instrument relative to the first surgical instrument in response to the path and the rotation of the flexible tether.

2. The surgical apparatus according to claim 1, wherein the shape sensor comprises an optical fiber sensor having a plurality of segments rotationally decoupled by the at least one rotation sensor.

3. The surgical apparatus according to claim 2, wherein the at least one rotation sensor interconnects adjacent segments of the plurality of segments and measures the rotation as a rotational relationship between the adjacent segments.

4. The surgical apparatus according to claim 1, wherein the at least one rotational sensor comprises a rotational linkage that rotationally decouples the flexible tether from at least one of the first surgical instrument and the second surgical instrument.

5. The surgical apparatus according to claim 1, wherein the at least one controller is configured to track the rotation identified by the rotation sensor from the at least one rotational sensor independent of the path of the shape sensor.

6. The surgical apparatus according to claim 1, wherein at least one of the first surgical instrument and the second surgical instrument is a manually manipulated device.

7. The surgical apparatus according to claim 6, wherein the manually manipulated device is manually oriented to a portion of a user.

8. The surgical apparatus according to claim 1, wherein the second surgical instrument moves freely relative to the first surgical instrument within a boundary defined by an extent of the flexible tether in connection with the first surgical instrument.

9. The surgical apparatus according to claim 1, wherein the first surgical instrument comprises a camera configured to capture image data in a field of view.

10. The surgical apparatus according to claim 9, wherein the at least one controller is further configured to:
identify a position and orientation of the surgical instrument in the field of view of the camera in response to the path and the rotation of the flexible tether.

11. The surgical apparatus according to claim 10, wherein the at least one controller is further configured to:
generate a graphic rendering of the surgical instrument projected in the field of view in response to the position and the orientation of the surgical instrument; and
output an augmented image feed to a display demonstrating the image data with the graphic rendering.

12. The surgical apparatus according to claim 1, wherein the second surgical instrument comprises at least one of a biter, grasper, retriever, pick, punch, hook, probe, elevator, retractor, a cutting blade or scissors.

13. The surgical apparatus according to claim 1, wherein the second surgical instrument is a surgical power tool comprising at least one of an ablation probe, a drill, a saw, a burr, a rasp, and a shaver.

14. A method for controlling a surgical imaging system comprising:
capturing image data with a camera in a field of view;
manually orienting a surgical device in connection with the camera via a flexible tether;
decoupling a path of the flexible tether from a rotation of the surgical device relative to the camera;
identifying the path of the flexible tether via path data;
identifying the rotation of the surgical device relative to the camera via rotation data; and
determining a position and an orientation of the surgical device relative to the camera in response to the path data and the rotation data.

15. The method according to claim 14, wherein the path is identified based on a plurality of rotationally decoupled path segments of the flexible tether.

16. The method according to claim 15, wherein the rotation is identified based on a rotational relationship between or among the rotationally decoupled path segments.

17. The method according to claim 16, wherein the rotation is identified by measuring a rotational relationship between adjacent portions of the rotationally decoupled path segments.

18.-22. (canceled)

23. A surgical imaging system comprising:
a camera configured to capture image data in a field of view;
a manually oriented surgical instrument;
a flexible tether interconnecting the camera to the surgical instrument, wherein the flexible tether comprises a plurality of orientation sensors configured to detect an orientation of the surgical instrument relative to the camera; and
at least one controller in communication with the camera and the flexible tether, wherein the controller is configured to:
identify a position and an orientation of the surgical instrument in response to orientation signals communicated from the orientation sensors;
identify at least one occluded portion of the surgical instrument in the image data;
generate a graphic rendering representative of the surgical instrument projected in the field of view in the position and the orientation of the surgical instrument;
selectively generate the augmented image feed with at least one graphic portion of the graphic rendering positioned over the at least one occluded portion; and
output an augmented image feed to a display demonstrating the image data

24. The imaging system according to claim 23, wherein the controller is further configured to:
identify whether the surgical instrument is positioned in the field of view based on the position and the orientation;
detect a presence of the surgical instrument in the image data; and
determine if the surgical instrument is occluded in the field of view based on the position and orientation of the surgical instrument and the presence of the surgical instrument in the image data.

25.-27. (canceled)

28. The imaging system according to claim 23, wherein the surgical instrument comprises a connected accessory of a plurality of interchangeable accessories, and wherein the controller is further configured to:
identify an accessory identification of the connected accessory;
in response to the accessory identification, access a graphic model of the connected accessory; and
generate the graphic rendering of the connected accessory based on the graphic model.

29.-37. (canceled)
